(19) United States
(12) Patent Application Publication
(10) Pub. No.: US 2019/0153373 A1
(43) Pub. Date: May 23, 2019

(54) Biofuel generation

(71) Applicant: Deepak Chaturvedi, Ajmer, Rajasthan (IN)

(72) Inventor: Deepak Chaturvedi, Ajmer, Rajasthan (IN)

(21) Appl. No.: 16/085,566

(22) PCT Filed: Mar. 2, 2017
(86) PCT No.: PCT/IB2017/051223

§ 371 Date: Sep. 15, 2018

(30) Foreign Application Priority Data
Mar. 15, 2016 (IN) 201611008996

(51) Int. Cl. C12M 1/00; C12M 1/33; C12M 1/36; C12M 3/00; C12P 7/64

(52) U.S. Cl. CPC C12M 21/12; C12M 29/24; C12M 45/02; C12P 2201/00; C12M 23/44; C12P 7/649; C12M 41/48

(57) ABSTRACT

The present invention provides a method for processing biomass, wherein the method includes sanitizing and grinding a biomass feedstock and reducing the ground feedstock into macromolecules. The feedstock reduction process further includes cooking the feedstock, pre-treating the cooked feedstock with at least one enzyme, and adding at least one pH controlling agent to the treated feedstock. The feedstock is analyzed at each reduction step for proper reduction and the reduction is controlled based on the analysis, wherein controlling the reduction includes diverting a flow of the feedstock based on the analysis. The macromolecules obtained by the reduction process are fermented, wherein macromolecules are extracted from each reduction step.

### CROSS REFERENCE TO RELATED APPLICATION

The present application is a U.S. national stage application (under 35 USC §§ 371) of PCT international application PCT/IB2017/051223 having an international filing date 2 Mar. 2017, which claims priority from Indian application No. 201611008996 filed with Indian Patent Office, Chennai on 15 Mar. 2016.

### TECHNICAL FIELD OF THE INVENTION

The present invention relates to a system and method for biofuel generation. More particularly, the present invention relates to a system and method for generating biofuel from ligocellulosic biomass through a multi-phase self-adaptive digestion process which allows controlling the digestion of biomass to increases a yield of biofuel in an eco-friendly manner.

### BACKGROUND OF THE INVENTION

From energy investment to energy generation, current processes including pretreatment (hydrolysis) of lignocellulosic biomass is one of the major challenges in production of low commercial grade biofuels. Going against nature, to have a faster digestion process, biomass is pretreated with matter of non-bio-organic matter and then pH, temperature and oxygen level is intelligently monitored for maximum yield. When these reactions occur at high speeds a tradeoff is made between maximum yield and time to yield maximum returns on investments as processed value added bio-chemicals.

Conversion of biomass to biofuels can be achieved by different methods that are broadly classified into thermal, chemical, and bio-chemical methods. As an example, biomass such as plants, can be converted into biofuels via a chemical and/or thermal process. As part of the conversion process, pre-treatment of biomass is required and can be done by several methods. Some of the exemplary methods of pre-treatment include:
1. Physical: chipping, grinding and milling, etc.
2. Physio-chemical: ammonia fiber explosion, recycling and percolation, CO2 explosion, ozonolysis, wetoxidation, etc.
3. Chemical: acid hydrolysis, dilute acid pretreatment, alkaline hydrolysis, organosolving, etc.
4. Biological: different fungi such as brown, white and soft rot fungi, etc.
   Many of these processes may use acids, bases (alkali), salts and gases to degrade biomass for extraction of value added bio-chemicals. In some cases, neutral pH liquid hot water or a diluted mixture with some of the above chemicals under varying temperatures of up to 350 degree Celsius and pressures of up to 1.6 MPa (16 bar) may be used.

A number of implementations are known that use synthetic agents for degradation and digestion of biomass and/or produce at high temperatures. Besides using synthetic agents, such implementations require complex equipment and feedstock specific agents for degradation and digestion. Addition of such chemicals to speed up degradation and digestion process leads to development of natural inhibitors to the process. Often, large amounts of additional synthetic agents may then be required to extract inhibitors during the refining process of required bio-chemicals. Such processes not only are expensive to manage but also entail harmful toxic waste recycling and management issues.

In one alternate implementation, a device to produce alcohol and bio fuels from bulky organic matter with a sea-based fermenter formed by a separating barrier made of flexible plastic film, and a solar-based distillation with vacuum assist is disclosed. The device includes a fermenter coupled to an inlet channel made of plastic film, converted to a continuous fermentation channel, a bio gas digester to generate methane, and a floating platform having an engine, pumps, a centrifuge, a sterilization unit, a crane and a water treatment unit, made of plastic film. Though inorganic chemicals are not used, the problem with this implementation is that the age reliability for commercial success of such a system is questionable. This is primarily because entire system when exposed externally to salty air and water may corrode rapidly and may likely cause operating and maintenance costs. Furthermore, such systems may not be used on large amounts of biomass available in areas far away from sea due to transportation, sanitation and other cost requirements.

U.S. Pat. No. 8,911,627 discloses a system and method for digesting a biomass through anaerobic digestion, wherein the biomass is passed through a series of digestion processes under different environment conditions. The digested output from each process is separated and undigested matter is forwarded to the next process. This method is applied in digesting the biomass for converting the biomass to methane or other bioproducts or biofuels, such as biogases, biosolids, safe fertilizers, and bio supplements.

Another U.S. Pat. No. 8,852,312 describes a system and method for biological treatment of biodegradable waste for producing products, such as rich biological/organic fertilizers, methane, and many useful byproducts. Similar to the method disclosed in U.S. Pat. No. 8,911,627, in this method, the feedstock is passed through multiple phases of digestion like sanitizing, grinding, heating, treating with enzymes, acidification etc. Even though the digestion is carried without using inorganic chemicals, the control over digestion process is not possible which may lead to emission of unwanted gases and affecting digestion efficiency.

Hence, there is need for a method and system for biofuel generation from biomass without using inorganic chemicals, while automatically controlling the digestion process based on the digestion progress and improving a yield of biofuel in an eco-friendly manner. Furthermore, there is need for a method for analyzing the digestion progress based on multiple parameters to determine the next phase of digestion.

### SUMMARY OF THE INVENTION

The present invention eliminates all the drawbacks of prior arts by providing a method for processing biomass, wherein the method includes sanitizing and grinding a biomass feedstock and reducing the ground feedstock into macromolecules. The feedstock reduction process further includes cooking the feedstock, pre-treating the cooked feedstock with at least one enzyme, and adding at least one pH controlling agent to the treated feedstock. The feedstock is analyzed at each reduction step for proper reduction and the reduction is controlled based on the analysis, wherein controlling the reduction includes diverting a flow of the feedstock based on the analysis. The macromolecules obtained by the reduction process are fermented, wherein macromolecules are extracted from each reduction step.

In one embodiment, the analysis of the feedstock is executed using optical based analysis of the feedstock particle size and osmotic pressure of the feedstock. If the particle size and pressure level are not within a corresponding threshold range, then the feedstock is determined as undigested and diverted for recycling or further reduction. In addition, conditions for reduction may also be adjusted based on the analysis. By this way the reduction process is automatically controlled based on the progress and a yield of biofuel is improved in an eco-friendly manner.

The present invention also provides a system for processing the biomass, comprising a grinding chamber, multi-chamber reduction unit, fermentation unit, analysis unit and a control unit. The analysis unit and control unit analyze and control the reduction process performed in the reduction unit to improve a yield of biofuel in an eco-friendly manner. The analysis unit includes a sensor unit to measure one or more parameters of the feedstock and the chambers, while the control unit controls one or more valves in each chamber for controlling a flow of the feedstock.

It is to be understood that both the foregoing general description and the following detailed description are exemplary and explanatory and are intended to provide further explanation of the invention as claimed.

### BRIEF DESCRIPTION OF THE DRAWINGS

The foregoing and other features of embodiments will become more apparent from the following detailed description of embodiments when read in conjunction with the accompanying drawings. In the drawings, like reference numerals refer to like elements.

FIG. 1 shows the flow diagram of the method for processing biomass in accordance with the first embodiment of the present invention.

FIG. 2 shows the block diagram of the system for processing biomass in accordance with the first embodiment of the present invention.

FIG. 3 shows the block diagram of the grinding chamber in accordance with the first embodiment of the present invention.

FIG. 4 shows the block diagram of the cooking chamber in accordance with the first embodiment of the present invention.

FIG. 5 shows the block diagram of the pre-treating chamber in accordance with the first embodiment of the present invention.

FIG. 6 shows the block diagram of the acidification chamber in accordance with the first embodiment of the present invention.

FIG. 7 shows the block diagram of the fermentation chamber in accordance with the first embodiment of the present invention.

### DETAILED DESCRIPTION OF THE INVENTION

Reference will now be made in detail to the description of the present subject matter, one or more examples of which are shown in figures. Each example is provided to explain the subject matter and not a limitation. Various changes and modifications obvious to one skilled in the art to which the invention pertains are deemed to be within the spirit, scope and contemplation of the invention.

The present invention eliminates all the drawbacks of prior art by providing method for processing biomass, wherein the method includes sanitizing and grinding a biomass feedstock and reducing the ground feedstock into macromolecules. The feedstock reduction process further includes cooking the feedstock, pre-treating the cooked feedstock with at least one enzyme, and adding at least one pH controlling agent to the treated feedstock. The feedstock is analyzed at each reduction step for proper reduction and the reduction is controlled based on the analysis, wherein controlling the reduction includes diverting a flow of the feedstock based on the analysis. The macromolecules obtained by the reduction process are fermented, wherein macromolecules are extracted from each reduction step.

The analysis of the feedstock is executed using optical based analysis of the feedstock particle size and osmotic pressure of the feedstock. If the particle size and pressure level are not within a corresponding threshold range, then the feedstock is determined as undigested and diverted for recycling or further reduction. In addition, conditions for reduction may also be adjusted based on the analysis. By this way the reduction process is automatically controlled based on the progress and a yield of biofuel is improved in an eco-friendly manner.

Biomass as referred to across the description includes without limitation any organic, non-fossilized material that is, or is derived from biological organisms, either living or dead. For example, biomass can be derived from waste generated from slaughter houses, fish and fish meal processing industries, municipal solid waste and industrial solid waste. Such wastes typically include for example, hides, skins, blood, rumen contents, bones, horns, hoofs, urinary bladder, gall bladder, uterus, rectum, udder, fetus, snout, ear, penis, meat trimmings, hide and skin trimmings, condemned meat, condemned carcass, esophagus, hair and poultry offal's (feathers, head). Biomass can be industrial cellulose or cloth waste. Biomass can also comprise additional components, such as proteins and lipids.

Alternately, biomass refers to cellulosic or lignocellulosic biomass material derived from plants, and includes material comprising macromolecules and optionally further comprising cellulose, hemicellulose, lignin, mono-, di-, oligo-, polysaccharides. In some embodiments macromolecules derived from biomass could be, liquids for example, lipids including fats, waxes, sterols, glycerides, phospholipids, and volatile fatty acids for example acetic, propionic, butyric and/or valeric acids.

Biomass includes, but is not limited to bioenergy crops, agricultural residues, and sludge from paper manufacture, yard waste, and wood and forestry wastes. Examples of biomass include, but are not limited to, algae, microalgae, corn grain, corn cobs, corn Stover, corn silage, coir pith, coir pith waste, groundnut shells or husk, grasses, wheat, wheat straw, hay, rice straw, waste paper, sugar cane bagasse, components obtained from processing of grains, trees, branches, roots, leaves, wood chips, sawdust, shrubs and bushes, vegetables, fruits, and flowers.

Biomass can be derived from a single source or can comprise a mixture derived from more than one sources. For example, biomass can comprise a mixture of material from multiple plant species, multiple hybrids or varieties of the same plant species, and multiple parts of a single plant species.

As used herein, the phrase “plant material” refers to all or part of any plant material that comprises simple and complex carbohydrates like lignocellulose, sugars, starches, lipids and/or other macromolecules that can be broken down into fermentable sugars. The plant material can be derived from a grain, fruit, legume, seed, stalk, wood, vegetable, root, or a part thereof. In some embodiments, the biomass comprises plant material derived from a plant selected from one or more of the group consisting of maize (i.e., corn), soybean, millet, milo, rye, triticale, oats, barley, rice, sorghum, Sudan grass, switch grass, miscanthus, alfalfa, cotton, sisal, hemp, jute, turf grass, rape (rapeseed), sunflower, willow, eucalyptus, poplar, pine, tobacco, clover, bamboo, flax, pea, radish, turnip, potato, sweet potato, cassava, taro, beet, sugar beet, ground nut, coconut and canola.

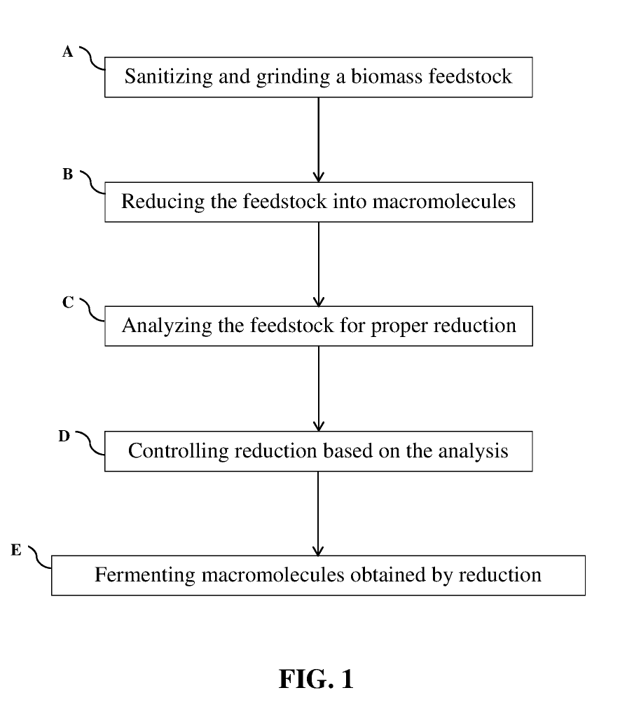

FIG. 1 shows the flow diagram of the method for processing biomass in accordance with the first embodiment of the present invention. The method initiates at step A by sanitizing and grinding a biomass feedstock to particle size. Feedstock may be a combination of various types as explained above and includes without limitation biomass feedstock for example, forestry, agricultural waste, industrial waste, organic municipal waste, slaughter house waste or any other bio degradable organic matter. Feedstock can be harvested using conventional harvesting equipment for the purpose of biofuel production or during the course of harvesting corn grain for other uses. Alternately, feedstock may be obtained by collecting biodegradable materials like municipal solid waste (sometimes called biodegradable municipal waste, or BMW) as green waste, food waste, paper waste, and biodegradable plastics. Other biodegradable wastes include human waste, manure, sewage, sewage sludge and slaughterhouse waste etc. In an embodiment, feedstock with specific amount of moisture content, say up to 99.5% may be preferred. In some embodiments, grinding may be done in the presence of water, microorganisms and/or enzymes. This enhances the pace of degradation of the feedstock. Microorganisms include without limitation bacteria, yeast, and fungi while enzymes include, for example, cellobiohydrolase, endoglucanase, xylanases, mannanase, hemicellulose, glycosidase, glycosyltransferase, lipase and amylase. It should be noted that the steps of shredding and grinding the feedstock may be performed in parallel or sequentially with shredding followed by grinding or vice-versa. Alternately, it is possible that one of the two steps—shredding or grinding—is used to degrade the feedstock.

While sanitizing, the feedstock is hydrated by continuously washing the feedstock with water, for example, purified water, in the first tank. A suitable ratio of water to the quantity of feedstock by volume is maintained in the first tank. As an example, a ratio of 1:1 to 1:100 may be used, where 1 is volume of feedstock and the other is volume of water. Optionally, based on the nature of the feedstock, microorganisms and/or enzymes may be added. The microorganisms/enzymes speed up the degradation of the biomass and thereby, the process of extraction of macromolecules. Microorganisms include without limitation bacteria and fungi while enzymes include for example, cellobiohydrolase, endoglucanase, xylanases, mannanase, hemicellulose, glycosidase, glycosyltransferase, lipase and amylase. Once the ground feedstock reaches a specific size i.e. 6 to 500 cubic micro meters or less, the feedstock slurry is forwarded to step B at which the slurry is reduced to macromolecules through various processes. The grinding process may be monitored via one or more of sensing of winding current, speed of grinder motor and/or real-time data from a network array of vibration sensors. For example, once motor current and/or noise, vibration and harshness levels are found to be below a predefined respective threshold and speed of the grinder motor is above a predefined respective threshold, it is deciphered that the feedstock has been evenly reduced to a required threshold size. In various embodiments, the grinding algorithms may vary based upon the type of feedstock.

In step B, the slurry is cooked at a specific temperature to reduce the slurry to macromolecules. The cooked slurry is analyzed for proper cooking in step C and if not cooked properly, then the slurry flow is diverted for cooking the slurry again in step D. Macromolecules are extracted from the slurry and forwarded for fermentation in step E. The remaining slurry is conveyed to for pre-treatment, wherein one or more enzymes are added to the slurry to facilitate microbial growth.

The enzyme is, for example, fresh cow dung, and is added at an exemplary ratio ranging from 1:5 to 1:100 (where 1 part is of the cow dung). The mixture is anaerobically recirculated internally for a retention time of up to for example, 72 hours. The temperature to be maintained is within the range of 5° C. to 60° C. to facilitate growth of microorganisms for further enzymatic hydrolysis under pressure greater than or equal to the atmospheric pressure for the range mentioned previously. Exact temperature is decided on the basis of the type of feedstock, microorganisms, etc. Temperature may be regulated via for example, solar thermal energy collection based systems for heating and exchanging heat with earth's infinite thermal inertia for cooling. This process of cooling saves huge amount of energy required for cooling the feedstock slurry and is in itself an incentive in the production process which is entirely organic. In some embodiments, for the entire duration of retention time, the feedstock slurry is continuously and softly mixed via a pressure pump. This action not only reduces the time of digestion but may also help in reducing flocculation. Further, in this stage, some water may be absorbed.

In step C, the pre-treated slurry is analyzed and macromolecules are separated from the slurry if the slurry is found to be pre-treated properly in step D. The remaining slurry is conveyed to for pH control. The macromolecules may contain simple and complex carbohydrates and Volatile Fatty Acids (VFA) such as acetic, propionic, butyric, valeric acids etc. The VFAs are separated by fractional distillation and/or ultrafiltration techniques, while the carbohydrates are extracted by using a separator or by any other electromechanical means that is within the teachings of the present invention. If the slurry is found to be not pre-treated properly, then the slurry is diverted back for pre-treatment in step D.

During pH control, the slurry, with a viscosity in a range between 200 to 40,000 centipoise, is mixed with acids to reduce or eliminate microbial organisms. The slurry is maintained at a temperature between 100 to 200° C. for a short duration for example, up to 20 minutes, to further hydrolyze the slurry or reduce unwanted microbial population. The acids may be hydrochloric acid or sulphuric acid with a concentration of about 0.0001M to 2M. If pH of the slurry is not within 2-7 and 7-10 during and after hydrolyses respectively, appropriate quantities of strong bases like quick lime or caustic soda are added. The base concentration may be 0.01% to 10%. Strong bases aid in formation/extraction of lignin from the slurry for further processing. The slurry is analyzed and macromolecules are extracted from the slurry, while the remaining slurry is conveyed for fermentation. The extract contains dissolved and/or colloid mixture of VFA's and carbohydrate. An appropriate cocktail of yeasts for example strains of saccharomyces cerevisiae, bacteria's such as zymomonas mobilis, enzymes such as Inulinase, Invertase, xylanase, amylase, zymase, and macro and micro nutrients such as ones present in used dry cells, parts of certain trees, etc. is mixed with extracts before conveying to fermentation. Gases such as methane, carbon dioxide and hydrogen sulphide are compressed and may be removed by dissolving in water under pressure and further reduced in concentration by passing over water absorbing microporous solids and/or gels like silica gel, activated alumina and/or zeolites to remove water vapor and passed over a suitable catalyst such as iron filings and calcium oxide to reduce hydrogen sulphide and carbon dioxide concentration and dried. This combustible mixture of gases may be stored/distributed/transported for further use at a pressure of for example, up to 200 bars at an atmospheric temperature range of say, up to 55° C. At step E, the extracts from the reduction process are mixed together and fermented to convert the macromolecules into a biochemical which is further processed to generate biofuel.

This process of analysis and control helps in understanding the progress of reduction based on multiple parameters to determine a next phase of reduction, and hence biofuel can be generated from biomass without using inorganic chemicals, while automatically controlling the reduction process based on the progress and also improving a yield of biofuel in an eco-friendly manner.

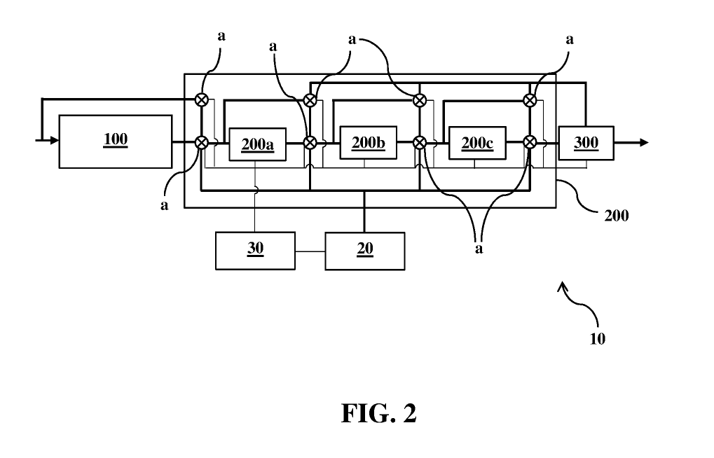

FIG. 2 shows the block diagram of the system for processing biomass in accordance with the first embodiment of the present invention. The system (10) includes a grinding chamber (100), a multi-chamber reduction unit (200), fermentation chamber (300), analysis unit (20) and a control unit (30). The feedstock is inputted to the grinding chamber (100) in which the feedstock is optionally water, microorganisms and/or enzymes and ground to a specific particle size. The ground slurry is conveyed to the reduction unit (200) which includes a cooking chamber (200a), pre-treatment chamber (200b), acidification chamber (200c) and a plurality of valves (a).

The slurry processed in each of the chambers (200a-200c) is analyzed in the analysis unit (20) with a set of sensing units coupled to the corresponding chambers (200a-200c). The analysis unit (20) transmits analysis information to the control unit (30) that is connected to each of the chambers (200a-200c) and the valves (a). The control unit (30) determines whether the slurry is properly reduced or not and controls the valves (a) and the chambers (200a-200c) accordingly. The control unit (30) operates the valves (a) to extract macromolecules and convey remaining slurry to next chamber, if the slurry is properly reduced. Otherwise, the slurry is fed back to the previous chamber.

For example, the analysis unit (20) analyzes the slurry from the cooking chamber (200a) and transmits the information to the control unit (30). The control unit (30) determines whether the slurry is properly cooked or not. If cooked properly, the control unit (30) operates the valves (a) to separate macromolecules from the slurry and convey the remaining slurry to the pre-treatment chamber (200b). If not cooked properly, the control unit (30) operates the valves (a) to direct the slurry back to the cooking chamber (200a) and adjusts one or more parameters of the cooking chamber (200a).

The control unit further controls the valves (a) to convey the extracted macromolecules to the fermentation chamber (300). The slurry from the acidification chamber (200c) is mixed with one or more reagents and transferred to the fermentation chamber (300). In the fermentation chamber (300), the slurry is mixed with the extracts and fermented to convert the mixture into biochemical that may be further processed to generate biofuel.

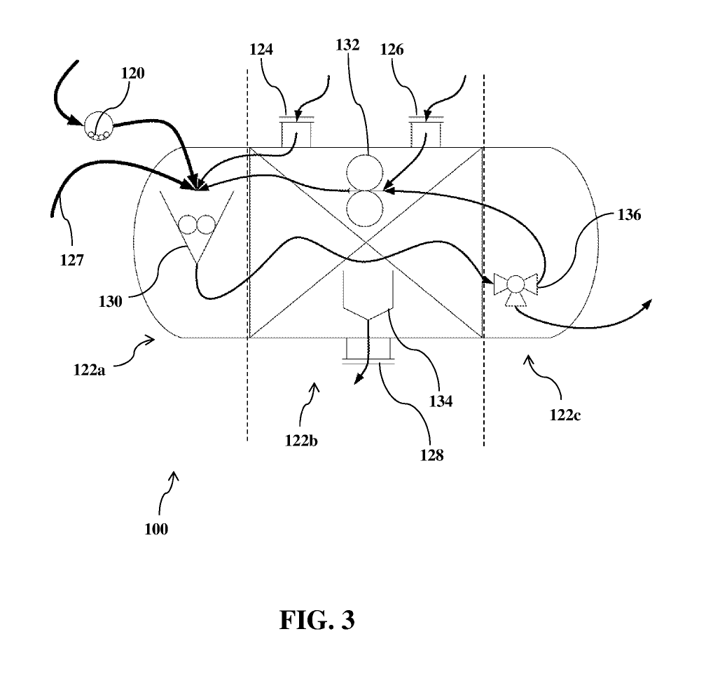

FIG. 3 shows the block diagram of the grinding chamber in accordance with the first embodiment of the present invention. The grinding chamber (200a) includes a first tank (122) divided into three parts (122a, 122b, 122c) and comprising two inlets (124, 126) and one outlet (128). A grinder (130) is provided in the first part (122a), while a mixer pump (132) and second tank (134) are provided in the second part (122b), and a valve (136) is provided in the third part (122c). The first tank (122) may be a conventional storage silo with large capacities and connected to one or more millers (120). In other embodiments, a silage chopper, crusher, breaker or ball roller machines may be connected to the tank (122) to reduce particle size of the feedstock.

The grinder (130) receives the shredded feedstock from the miller (120) and optionally water, microorganisms and/or enzymes from the inlets (124, 126) respectively. The presence of water, microorganisms and/or enzymes may enhance the grinding process. The grinder (130) is a wet stone grinder that includes at least two grinding wheels stacked upon one another. Interaction between the two grinding wheels is entirely based on mechanical friction. A motor rotates a first wheel which in turn grinds the feedstock against the second wheel. The second wheel sits on top of the first wheel and maintains an axis in an aligned confinement of rotation.

The second tank (134) is provided at the bottom of the second part (122b) and is responsible for carrying out cleaning operation. Though the second tank (134) is shown situated at the bottom and inside the first tank (122), the second tank (134) can be connected externally to the first tank (122). In the second tank (134), unwanted debris such as stone, plastics, metal and soil impurities are removed from the feedstock slurry via sedimentation, filtering particle size using selective sieves in combination with electro-mechanical pressing and/or centrifuging means, etc. Further, heavy and dense impurities like metal and sand are trapped via such sieves that provide for cleaning/expulsing of these impurities. Alternately, to perform cleaning operation, the second tank (134) may be temporarily disconnected for manual removal and cleaning of trapped impurities. Alternately, auto-chute mechanisms may be used for removing trapped impurities.

The feedstock slurry from the second tank (134) is fed to the third part (122c) to check if the particle size of the slurry is reduced to the desired size. The third part (122c) contains a valve (136) that may be connected to a microprocessor based electronic control unit that controls the opening and closing of the valve (136). The electronic control unit may be coupled to one or more sensors that determine whether the feedstock is ground to a desired level and accordingly opens the valve (136) to push the feedstock slurry to the next tank else the valve (136) remains in closed state for recycling the feedstock slurry to the grinder (130) for further grinding operation. The valve (136) may be a pressure valve.

In some embodiments, the mixer pump (132) is provided in the second part (122b) to mix the feedstock slurry, water and/or microorganism/enzymes through a churning action.

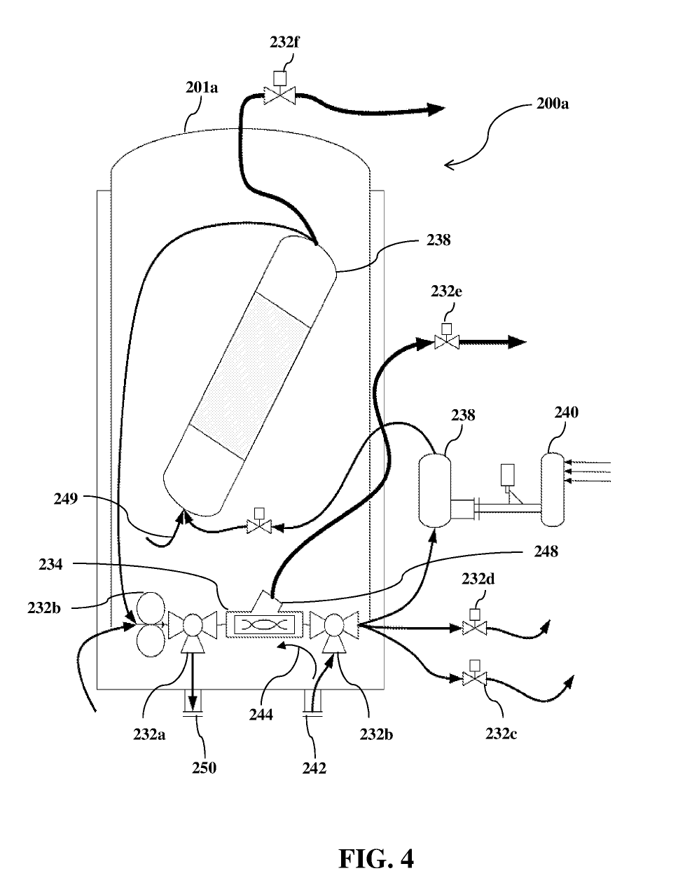

FIG. 4 shows the block diagram of the cooking chamber in accordance with the first embodiment of the present invention. The cooking chamber (200a) comprises a third tank (201a) enclosing a pump (230), a plurality of valves (232a-232g), one or more bio-membrane reactors (234), duct (236), holding column (238), and an optical density measurement (ODM) system (240). The pump (230) is connected to the valve (232a) which in turn is connected to the bio-membrane reactor (234) to which the valve (232b) is connected. The pump (230) may be a booster or pressure pump that increases the flow rate of the feedstock as the feedstock passes through the pump (230).

The valves (232a-232g) in general are provided to control the flow direction and process pressure of the slurry passing through various stages of the digestion process. The valves (232a-232g) may either stop the flow or allow controlled passage of the slurry. The valves (232a-232g) are operated by the control unit (30) based upon the information from the analysis unit (20). For example, the valve (232a) may be operated to change flow direction for membrane regeneration through back washing when all the feedstock has been processed in the cooking chamber (200a). This could be done based on the ODM system (240) which is of the sensing units of the analysis unit (20). In another example, the valve (232b) may allow a passage channel in the direction from an inlet (242) to the bio-membrane reactor (234) as indicated via arrow (244) or from valve (232b) to the holding column (238) as indicated via arrow (246). In yet another example, the valve (232c) is opened to allow unprocessed feedstock slurry to be passed to the first tank (122) for further grinding, enzymatic hydrolysis and reprocessing. The valve (232d) may be opened to allow the slurry to be passed to the fermentation chamber (300). The valve (232e) may be opened to collect macromolecule rich water extracted from the bio-membrane reactor (234). The valve (232f) may be opened to collect steam condensate from the duct (236). The valve (232g) may be opened to pass the slurry from the holding column (238) to the duct (236). Further, in various embodiments, lesser or more number of valves may be used as required to control the process.

The bio-membrane reactor (234) contains one or more bio-membranes to extract macromolecules for example, dissolved and/or suspended colloids/oil emulsions in liquid state in the slurry. The bio-membranes extract the macromolecules via one or more of filtration, microfiltration, nanofiltration, ultrafiltration, etc. The bio-membranes present in the reactor (234) may be of varying mesh sizes for example from 5 Angstrom to 10,000,000 (107) Angstroms.

The bio-membrane reactor (234) continuously absorbs water rich in dissolved carbohydrates. Typically, the bio-membrane reactor (234) is located between the pressure pump (230) and pressure regulator valve (232b). The bio-membrane reactor (234) may further include one or more plated oblong inclined or planar ducts (248) to aid in easy removal of gases and/or dissolved carbohydrates. Absorbed carbohydrate rich water may be continuously transferred to a separate continuous fermentation, distillation and extraction area optimized for generation of oil and alcohol based biofuels such as bioethanol and biodiesel. Recycled water may be continuously added and the process may be continued until dissolved carbohydrate concentration is less than 1%. In some embodiments, the bio-membrane reactor (234) may be made inclined to reduce membrane fouling due to expelled water vapor and gases during heating under pressure.

In an embodiment, to prevent and/or reduce fouling and clogging of the bio-membrane, the bio-membrane is submerged inside the slurry. The third tank (201a) is provided with a back washing system that is coupled to the bio-membrane reactor (234). Through an inlet (242) of the back washing system, a cleansing agent, for example, diluted tartaric or citric acid derived from natural citrus fruits is passed through the bio-membrane. This process could be followed by washing with purified water to expel out cleansing agent. Cleansing agent regenerates bio-membrane and also removes any impurities that may be attached or clogged into the bio-membrane while dealing with the feedstock slurry. The cleansing agent is then expelled from an outlet (250) of the back washing system. In some embodiments, the back washing system may be designed to prevent back washed liquid from getting mixed with liquid or gaseous feedstock in the cooking chamber (200a) and to expel the washed liquid completely after membrane regeneration process. In some other embodiments, the backwashed liquid could be reused or recycled in later stages for example, to alter pH of feedstock or further aid in digestion process.

The duct (236) may be inclined duct imitating flow in an Upflow Anaerobic Sludge Blanket Digester (UASB) used to heat the feedstock slurry. The duct (236) has a provision for exposing the slurry to a continuously interacting steam in the stirring and moving feedstock at 120° C. and up to 1.2 bar pressure. Alternately, the feedstock slurry may be heated at the aforesaid temperature and pressure conditions. In some embodiments, the duct (236) may be horizontally mounted such that easy removal of gases and water vapor trapped in the feedstock slurry is ensured.

When smaller quantities of feedstock of varying nature such as sugar rich fruit pulp, cellulose and lignin rich wood waste, lipid and protein rich algae and/or slaughter house waste are fed in the batches. Amount and type of available bio chemical in each stage may be such that, further continuous processing of extract is not process efficient. If, the extract volume is lower than designed process size, it makes sense to hold the extract in a column for further batch process. The holding column (238) is optionally used to stock the extract for further batch processing in a gated array of stage and extract specific holding columns. In some embodiments, the gated arrays may optionally be connected in a non-exclusive, stage and extract specific manner. The extracts from each chambers are temporarily held in the corresponding holding columns and then fed to the fermentation tank.

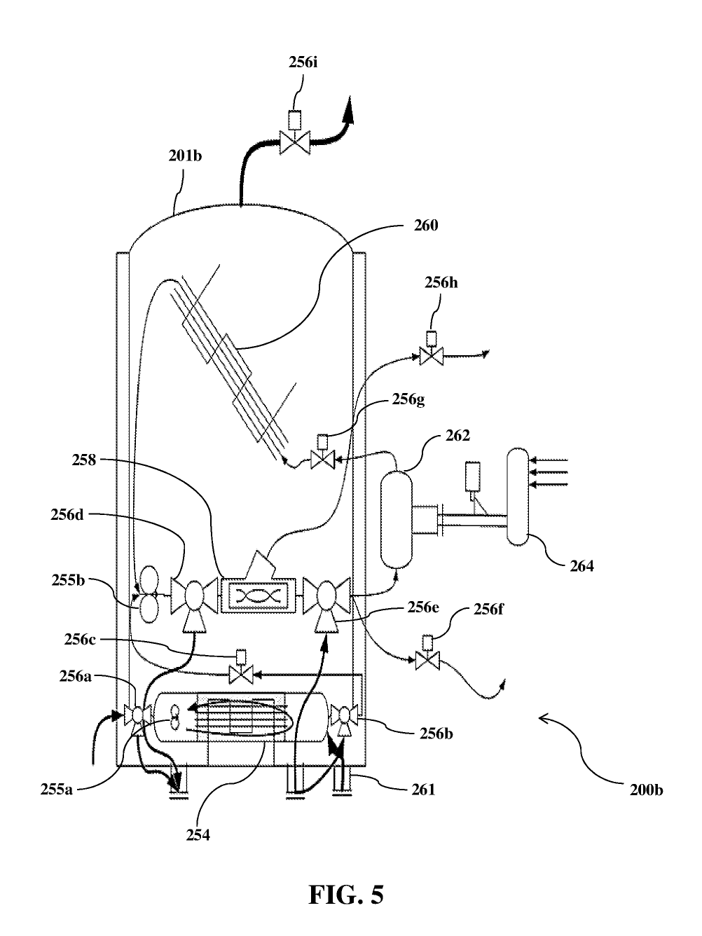

The ODM system (240) is used to analyze the feedstock slurry, wherein a sample quantity of feedstock say 1 mL, which is diluted with distilled water with up to 1,000 parts per million or more, is taken in a column and low cost reagents are added, for example biuret for proteins, Sudan red for lipids, benedict and iodine for simple and complex carbohydrates. The readings of pH values are taken and accordingly one or more of the valves (232c-232e) are opened. The valve (232e) is used to extract dissolved and/or suspended colloids of macromolecules as explained earlier. If the macromolecule concentration is found high but estimate of particle size of feedstock is more than required to pass to next stage. The valve (232d) is opened to recycle the feedstock to the first tank (122) via the inlet (127). If all the feedstock has been ground below earlier mentioned thresholds and concentration of dissolved and/suspended colloids of macromolecules is also estimated to be below thresholds, the valve (232c) is opened to pass spent feedstock to the fourth tank (201b) of the pre-treatment chamber (200b) illustrated in FIG. 5.

The fourth tank (201b) includes an enzymer and heat exchanger (254), a plurality of pumps (255a-255b), a plurality of valves (256a-256i), a bio-membrane (258), a tube heat exchanger (260), a closed column (262), and an ODM system (264). The feedstock slurry fed from the third tank (201a) to the enzymer and heat exchanger (254) is treated with enzymes and thereafter heated. The slurry may be mixed repeatedly via the pump (255a). The enzymes may be fed from the inlet of an enzyme adder flange (261) connected to the heat exchanger (254). The enzyme and heat exchanger (254) is a long tube horizontally placed at bottom of the fourth tank (201b) and facilitates exchange of heat with earth using earth's inertia for cooling.

Once the slurry is processed in the enzymer and heat exchanger (254), the valves (256b, 256c) are opened to pass the slurry to the mixer pump (255b) for continuous mixing. The macromolecules released due to heating and mixing operations are absorbed through the bio-membrane reactor (258) and obtained via the valve (256h) and passed to the fermentation chamber (300). The ODM system (264) analyzes the slurry to determine the concentration of macromolecules in the feedstock in order to decide whether the feedstock is ready for the next stage or should be treated further in the fourth tank (201b). For further processing, feedstock is fed to the heat exchanger (260) which facilitates controlling the temperature of the feedstock.

To be able to digest a variety of feedstock, in addition to process pH and pressure, precise control of temperature is required in every stage of digestion process. Based on the nature of feedstock, optimal temperature ranges are maintained through a connected bio-informatics cloud for efficient and fast micro-organic activity. Psychrophilic (less than 20° C.), Mesophilic (20° C. to 45° C.) or thermophilic (greater than 41° C.) micro-organism may be used to speed up bio-digestion of the feedstock. Thermal fluids like hot oil and/or ethylene glycol may be used to increase or decrease the process temperature by exchanging heat via these thermal fluids through concentrating solar thermal energy collection system or via infinite thermal inertia of underground earth.

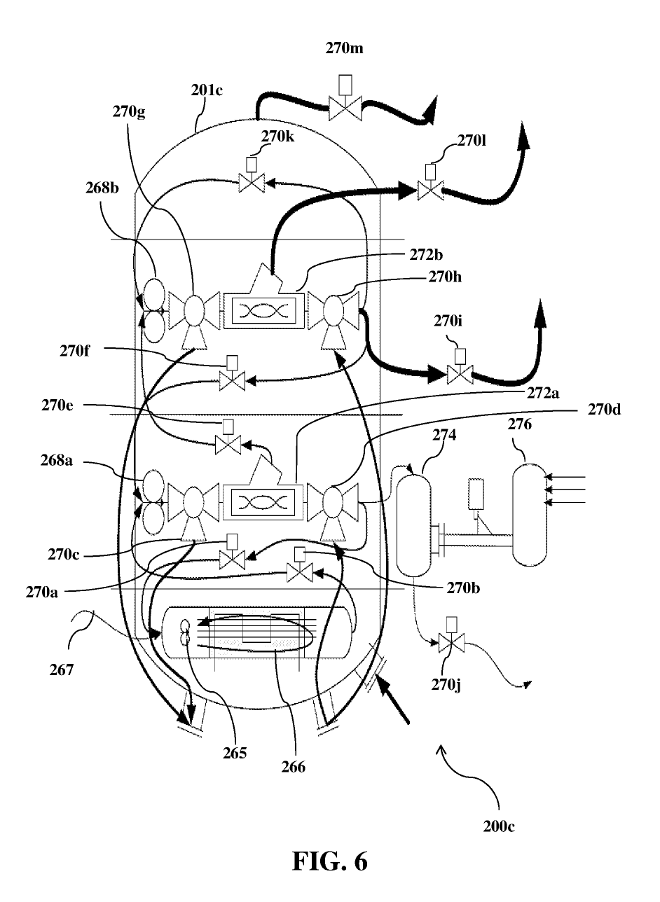

FIG. 6 shows the block diagram of the acidification chamber in accordance with the first embodiment of the present invention. The acidification chamber (200c) includes a fifth tank (201c), wherein partially digested feedstock is introduced from the pre-treatment chamber (200b) via an inlet (267). A stirring mixer (265) and heat exchanger (266) along with multiple stages of macromolecule extraction are provided in the fifth tank (201c). A mixer pump (268a) is connected to a valve (270c) which is connected to a bio-membrane reactor (272a) followed by another valve (270d) to extract macromolecules. It is possible to include more stages of macromolecule extraction, wherein each stage will be responsible for extraction of macromolecules of predefined size. In some embodiments, the mesh size of the bio-membrane of the reactor (272a) may be coarser than the mesh size of the bio-membrane of the reactor (272b). Depending on the mesh size, macromolecules are extracted via corresponding valves (270l, 270i) while the feedstock slurry is recycled back to the exchanger (266) for further reduction/digestion.

A first bio-membrane (272b) may be used for reverse osmosis or filtration of particles size of up to a maximum of 50 Angstrom (for example ions and sugars), a second bio-membrane (272a) for filtering out particle size from 10 to 2000 Angstrom (for example proteins and enzymes), a third bio-membrane (not shown) for filtering particle size from 500 to 20000 Angstrom (for example oil emulsions and colloids), and a fourth bio-membrane (not shown) for filtering particle sizes from 10000 Angstrom to 10,000,000 Angstrom (for example certain bacteria and yeast cells, sand and other debris). The bio-membranes may be made of polymers like nitrocellulose, cellulose acetate, cellulose esters, polyether sulfone, polyacrylonitrile, polyamide, polyimide, polyethylene, polypropylene, polytetrafluro ethylene, polyvinylidene fluoride, or polyvinyl chloride.

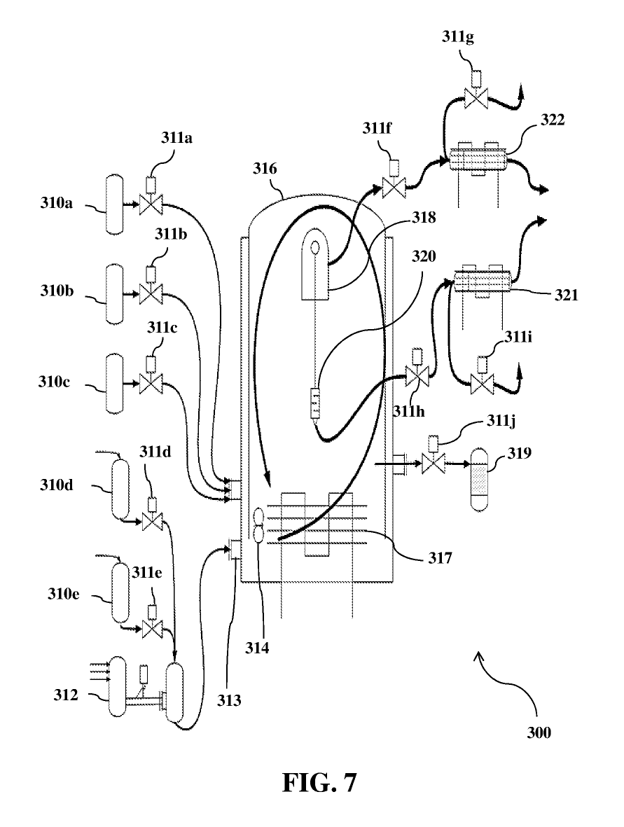

FIG. 7 shows the block diagram of the fermentation chamber in accordance with the first embodiment of the present invention. The fermentation chamber (300) processes macromolecules extracted from the tanks (201a-201c) through valves (232e, 256h, 270l, 270i) and the holding columns (310d, 310e). Primary method of biofuel extraction employed is batch fed fermentation and continuous extraction. Determination of type and concentration of feedstock is estimated via ODM. Based on the determination, reagents and catalysts stored in the holding tanks (310a-310c) are added by activating gate valves (311a-311c) into a fermentation tank (316) where the extracted macromolecules are transferred in an extraction flange (313). The mixture is recirculated over a heat exchanger (317) by a mixer pump (314) for a fermentation period. Continuous extraction is done by passing evaporated mixture over an oil separator (318) and fractionating column (320). Fractional distillation is carried out by controlling temperature of the heat exchanger (317) utilizing solar and underground means as explained earlier. As an alternative to oil separators, purifiers, clarifiers and/or distillers, one or more bio-membrane reactors may be employed to extract volatile fatty acids (VFA) and alcohols from the steam condensate through fractional distillation and/or ultrafiltration techniques.

Enriched distillate extracts of lipid origin are further passed through a tube membrane separator (322). Enriched distillate extracts of alcoholic origin are further passed through a tube membrane separator (321). The membrane separators (321, 322) are water absorbing membranes like silica gel, activated alumina, synthetic and/or natural zeolites. In some embodiments, liquids like ethylene glycol and/or glycerol are also used. In some embodiments, complete water absorption (water content less than 0.2%) for generation of anhydrous ethanol from this distillate is done through pervaporation and vapor permeation. In this process, optionally, salts like sodium chloride, calcium chloride can be added to liquid desiccants to increase the desiccation efficiency and reduction in the uptake of desiccant. These tubes and water absorbing materials are subsequently regenerated by heating through solar energy. Condensed water extracted from the steam can be recycled for use in the process again through gate valves (311g, 311i). Permeate extraction is made possible by creating a partial pressure across the water absorbing membrane using negative pressure created by vacuum pumps and/or positive pressure by using centrifuge. To accommodate volumetric change in the size of water absorbing membrane, a provision may exist to install these tube columns and/or control flow of alcohol rich permeate vertically such that membranes can expand in the direction of negative pressure.

In an exemplary embodiment, steam generated in the above membrane regeneration process at valves (311g, 311i) may be recirculated into the third tank (201a) at input (249) to aid in digestion of virgin feedstock. In each chamber (200a-200c), the reduction/digestion process is analyzed through various means such as ODF, pressure measurement etc., which helps in understanding the progress of reduction based on multiple parameters to determine a next phase of reduction, and hence biofuel can be generated from biomass without using inorganic chemicals, while automatically controlling the reduction process based on the progress and also improving a yield of biofuel in an eco-friendly manner.

It is to be understood, however, that even though numerous characteristics and advantages of the present invention have been set forth in the foregoing description, together with details of the structure and function of the invention, the disclosure is illustrative only. Changes may be made in the details, especially in matters of shape, size, and arrangement of parts within the principles of the invention to the full extent indicated by the broad general meaning of the terms in which the appended claims are expressed.

I claim:

1. A method for processing biomass, the method comprising:
sanitizing and grinding a biomass feedstock;
reducing the ground feedstock into macromolecules, wherein the reducing the feedstock includes:
a. cooking the feedstock;
b. pre-treating the cooked feedstock with at least one enzyme;
c. adding at least one pH controlling agent to the treated feedstock;
analyzing the feedstock at each reduction step for proper reduction;
controlling the reduction based on the analysis, wherein controlling the reduction includes diverting a flow of the feedstock based on the analysis;
fermenting macromolecules obtained by reduction of the feedstock, wherein macromolecules are extracted from each step of the reduction.

2. The method as claimed in claim 1, wherein the step of diverting includes directing an unreduced portion of the feedstock for:
i. cooking the feedstock;
ii. pre-treating the cooked feedstock with at least one enzyme; or
iii. adding at least one pH controlling agent to the treated feedstock.

3. The method as claimed in claim 1, wherein the step of diverting further includes forwarding a digested portion of the feedstock to the fermentation chamber.

4. The method as claimed in claim 1, wherein the step of analyzing includes measuring at least one parameter of the feedstock.

5. The method as claimed in claim 1, wherein the step of analyzing includes measuring at least one condition of performing one of the reduction steps.

6. The method as claimed in claim 5, wherein the step of analyzing includes determining that the feedstock is undigested if the measured parameter is not within a threshold range.

7. A system for processing biomass, the system comprising:
at least one grinding chamber (100) for sanitizing and grinding a biomass feedstock;
a multi-chamber digestion unit (200) for reducing the ground feedstock;
wherein the digestion unit (200) includes:
a. at least one cooking chamber (200a) for cooking the feedstock;
b. at least one pre-treating chamber (200b) for pre-treating the cooked feedstock with at least one enzyme;
c. at least one acidification chamber (200c) for adding at least one pH controlling agent to the treated feedstock;
d. at least one valve (a) in each chamber for controlling a flow the feedstock;
at least one fermentation chamber (300) for fermenting macromolecules from each of the chambers (200a-200c), wherein macromolecules are extracted at the end of each of the chambers (200a-200c) and fed to the fermentation chamber (300);
at least one analysis unit (20) for analyzing the feedstock at each of chambers (200a-200c) for proper reduction;
at least one control unit (30) for operating the valve (a) for controlling the reduction process in the digestion unit (200) based on the analysis, wherein controlling the reduction process includes diverting the flow of feedstock based on the analysis.

8. The system as claimed in claim 7, wherein the control unit (30) diverts an unreduced portion of feedstock towards the cooking chamber (200a), pre-treatment chamber (200b) or acidification chamber (200c).

9. The system as claimed in claim 7, wherein the control unit (30) diverts the macromolecules towards the fermentation chamber (300).

10. The system as claimed in claim 7, wherein the analysis unit (30) includes at least sensing device for measuring at least one parameter of the feedstock.

11. The system as claimed in claim 7, wherein the analysis unit (30) includes at least one sensing device for measuring at least one parameter of at least one of the chambers (200a-200c).

12. The system as claimed in claim 11, wherein the control unit (30) determines that the feedstock is undigested if the measured parameter is not within a threshold range.
